(19) United States
(12) Patent Application Publication
(10) Pub. No.: US 2008/0230422 A1
(43) Pub. Date: Sep. 25, 2008

(54) Surgical data carrier

(75) Inventors: Thomas Pleil, Bad Duerrheim (DE); Markus Nesper, Tuttlingen (DE); Dirk Schauer, Berlin (DE); Dieter Weisshaupt, Immendingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 12/070,057

(22) Filed: Feb. 13, 2008

(30) Foreign Application Priority Data
Feb. 28, 2007 (DE) 10 2007 011 093

(51) Int. Cl. B65D 85/00; A61B 17/80

(52) U.S. Cl. 206/438; 606/280

(57) ABSTRACT

The invention relates to a surgical data carrier for the identification of a medical implant, in particular, a surgical plate for the fixing of bones or bone fragments, wherein the data carrier has a carrier element and a connecting device which comprises a receptacle for the insertion of at least part of the implant. In order to make such a surgical data carrier available which has an improved handling capability, it is suggested in accordance with the invention that the data carrier have an actuating device which can be actuated by a user and with which the connecting device can be transferred from a connecting position, in which the implant is held in the receptacle, into a release position, in which the data carrier can be detached from the implant. The invention relates, in addition, to a surgical implantation system.

The present disclosure relates to the subject matter disclosed in German application number 10 2007 011 093.8 of Feb. 28, 2007, which is incorporated herein by reference in its entirety and for all purposes.

### BACKGROUND OF THE INVENTION

The invention relates to a surgical data carrier for the identification of a medical implant, in particular, a surgical plate for the fixing of bones or bone fragments, wherein the data carrier has a carrier element and a connecting device which comprises a receptacle for the insertion of at least part of the implant.

In addition, the invention relates to a surgical implantation system comprising at least one medical implant, in particular, a surgical plate for the fixing of bones or bone fragments and a surgical data carrier which can be detachably connected to the implant for the identification of the implant, wherein the data carrier has a carrier element and a connecting device which comprises a receptacle for the insertion of at least part of the implant.

In the following, the surgical data carrier will also be designated simply as “data carrier”, the surgical implantation system simply as “implantation system”.

A surgical data carrier of the type specified at the outset is known from U.S. Pat. No. 6,929,646 B2. It is part of a surgical implantation system which comprises, apart from the data carrier, a surgical plate for the fixing of bones or bone fragments. The data carrier bears data identifying the implant. For handling the implant, the data carrier has a carrier element which serves as a handle for the implant and on which a connecting device is arranged. This is in the form of two gripping arms. The gripping arms define between them a receptacle for an attachment lobe which is arranged on the implant, is to be screwed to the bone or bone fragment and is held first of all in the receptacle during use of the implantation system. In order to separate the data carrier from the implant, a relative movement of data carrier and implant is required. Both the implant and the data carrier are to be acted upon with the pulling forces necessary for bringing about the relative movement. In this respect, there is the risk of the gripping arms, which are of a filigree design, breaking off, in particular, when the attachment lobe becomes wedged in the receptacle during detachment of the data carrier. As a result, not only is the possibility excluded, on the one hand, of the data carrier being connected to the implant again. On the other hand, the gripping arm can remain in the body of the patient and represent a potential risk of infection. As a result, the health of the patient can possibly be impaired to a considerable degree.

Therefore, it would be desirable to have a surgical data carrier and a surgical implantation system of the type specified at the outset with an improved handling capability.

### SUMMARY OF THE INVENTION

In accordance with the invention, it is suggested in a surgical data carrier of the generic type, that the data carrier has an actuating device which can be actuated by a user and with which the connecting device can be transferred from a connecting position, in which the implant is held in the receptacle, into a release position, in which the data carrier can be detached from the implant.

It is possible for the user, by means of the actuating device, to carry out the transfer of the connecting device from a connecting position into a release position in a user-friendly and controllable manner. This improves the handling capability of the data carrier. Furthermore, the solution to the object according to the invention allows the user to intentionally delay the removal of the data carrier from the implant for such a time until the connecting device has taken up a release position. It is, therefore, possible for the user to control the taking up of the release position, i.e., the releasability of the data carrier. The risk of damage to the data carrier, which can occur, for example, when the data carrier is detached from the implant too quickly or in an inappropriate or uncontrolled manner, is consequently reduced.

It is favorable when the connecting device is designed in such a manner that it can be transferred from the connecting position into the release position without any forces acting on the implant. This gives the data carrier an even better handling capability. Solely forces acting on the data carrier can, therefore be sufficient to transfer the connecting device from the connecting position into the release position. Subsequently, the data carrier can be detached from the implant due to a relative movement thereof. In contrast thereto, it has proven to be disadvantageous when using the implantation system described in the cited publication U.S. Pat. No. 6,929,646 B2 that a relative movement of data carrier and implant can be accomplished only when pulling forces act on both the implant and the data carrier. This is particularly disadvantageous in the case of implants which have small dimensions and are, therefore, difficult for the user to grasp. These include, in particular, surgical plates for the fixing of bones and/or bone fragments which are used in the field of oral, dental or facial surgery.

In the connecting position, the implant can be held in the receptacle, for example, in a force-locking, form-locking and/or frictional manner. It is, for instance, possible for a section of the implant to be partially enclosed in an annular manner by the connecting device. This can form a recess for this purpose and/or engage around or behind the implant in sections. It is also conceivable for part of the implant to be held in the receptacle by way of clamping.

The receptacle is preferably enlarged during the transfer of the connecting device from the connecting position into the release position. As a result of the enlargement of the receptacle during the transfer of the connecting device into the release position, any force locking, form locking and/or frictional locking between the data carrier and the implant can, for example, be overcome in order to detach the data carrier from the implant. The receptacle favorably has an insertion opening for the implant which is likewise enlarged when the connecting device is transferred from the connecting position into the release position. The insertion opening can advantageously be enlarged to such an extent that the part of the implant held in the receptacle can pass through the insertion opening during the detachment of the data carrier from the implant without having to touch the sections bordering on the insertion opening.

It is favorable when the connecting device comprises at least one connecting element which limits the receptacle at least in sections and which interacts with the implant for the connection of the data carrier.

A particularly simple construction of the data carrier can be achieved when the carrier element comprises the at last one connecting element.

The same advantage can be achieved alternatively with an embodiment of the data carrier, with which the at least one connecting element is arranged on the carrier element.

The at least one connecting element preferably has a contact surface which abuts on the implant in the connecting position. A form-locking, force-locking or frictional connection can be provided, for example, between the connecting device and the implant in the connecting position by means of the contact surface. It can be ensured by a form-locking, force-locking and/or frictional connection that the implant is held securely in the receptacle in the connecting position. Any wobbling of the implant in the receptacle can be avoided in this way.

In order to make a simple construction of the data carrier possible, the connecting device advantageously comprises two or more connecting elements which limit the receptacle in sections. The receptacle can, in particular, be defined between them. Each connecting element limits the receptacle at least in sections. Furthermore, it may be provided for each of the connecting elements to have a contact surface which abuts on the implant in the connecting position.

The two or more connecting elements are preferably movable relative to one another. As a result of a relative movement of the two or more connecting elements, it is possible in a technically simple manner for the receptacle and/or its insertion opening to be enlarged so that the detachment of the data carrier from the implant is made possible. The transferability of the connecting device from the connecting position into the release position and/or vice versa can be based, in particular, on the relative movement of the two or more connecting elements. This relative movement can be initiated, for example, by means of the actuating device. When two or more connecting elements are present, it is possible for all or only some of the respective connecting elements to be movable relative to one another.

As already mentioned, the at least one connecting element can be arranged on the carrier element. It is favorable when it is arranged or mounted on the carrier element so as to be movable since, as a result, the at least one connecting element may be designed to be movable in a technically simple way. A movable mounting of the at least one connecting element may be brought about in accordance with the methods known to the person skilled in the art. A movable arrangement of the at least one connecting element on the carrier element may be achieved, for example, by way of a materially elastic section of the at least one connecting element or a materially elastic intermediate member between the carrier element and the at least one connecting element.

The at least one connecting element is preferably designed to be deformable at least in sections and, even more preferably, even elastically deformable. As a result of deformation of the at least one connecting element which can be based on a material elasticity thereof, it is possible, for example, for the at least one connecting element to be movable at least in sections. It may be provided for the at least one connecting element to be secured to the carrier element with its deformable section. The deformable section of the at least one connecting element can, in particular, form a film hinge, with which the at least one connecting element is arranged on the carrier element so as to be pivotable. In accordance with the preceding explanations, the transferability of the connecting device from the connecting position into the release position and/or vice versa can be based on such a deformability of the at least one connecting element.

The at least one connecting element is advantageously designed as a snap-in element. As a result, a form-locking connection can, for example, be provided between the at least one connecting element and the implant, by way of, for example, engagement around or behind the implant. This allows the implant to be held particularly securely in the receptacle in the connecting position. The implant can also have a suitable snap-in receptacle for the snap-in element.

It has proven to be favorable when the at least one connecting element is designed as a clamping element. This offers the possibility of providing a force-locking or frictional connection between the at least one connecting element and the implant in the connecting position so that, as a result, the implant is held securely in the receptacle in the connecting position.

If two or more connecting elements are present, it may, of course, be provided for connecting elements to be used which are designed not only as clamping elements but also as snap-in elements.

The connecting device preferably has a securing member which secures the implant in the receptacle in the connecting position. In this respect, it is favorable when the securing member is arranged on the at least one connecting element. It can be ensured as a result of the securing member that the implant is held particularly securely in the receptacle in the connecting position. A projection of the at least one connecting element, which forms a recess for the implant in the connecting position and/or engages around or behind it, may be provided, for example, as securing member.

It is particularly favorable when the securing member can be brought into engagement with a receptacle comprised by the implant since, as a result, it is possible for the implant to be held even more reliably in the receptacle in the connecting position. A securing pin which is arranged on the at least one connecting element and engages in a receptacle of the implant is, for example, conceivable. Alternatively, the securing member can be designed, for example, as a snap-in nose. This can interact with a snap-in receptacle arranged in the implant.

The actuating device advantageously has at least one actuating member for moving at least one connecting element comprised by the connecting device. As a result of actuation of the actuating member by the user, at least one connecting element can be moved in this way. As a result of the movement of at least one connecting element, the receptacle for the implant can, as explained above, be enlarged so that the data carrier can be detached from the implant. The connecting device can, therefore, be transferred by actuation of the at least one actuating member from a connecting position into a release position.

It may be provided for an actuating member to be associated with each connecting element; it is, however, also possible for several connecting elements to be movable by means of one actuating member.

In a particularly preferred embodiment of the data carrier according to the invention, the at least one actuating member comprises the at least one connecting element. It may be provided, for example, for the at least one actuating member to have a free end which forms the at least one connecting element. If the at least one actuating member is actuated, for example, moved, the at least one connecting element can also be moved with it. One end of the at least one actuating member facing away from the at least one connecting element can be arranged, for example, on the carrier element. This favors a simple construction of the data carrier.

Altogether, it is of advantage for achieving a simple construction of the data carrier when the at least one actuating member is arranged on the carrier element.

The at least one actuating member is preferably arranged or mounted on the carrier element so as to be movable. As a result, the at least one actuating member and, where applicable, a connecting element arranged on it can be designed to be movable in a technically simple manner. A movable mounting of the at least one actuating member can be brought about in accordance with the methods known to the person skilled in the art. A movable arrangement of the at least one actuating member on the carrier element can be brought about, for example, by a materially elastic section of the at least one actuating member or a materially elastic intermediate member between the carrier element and the at least one actuating member.

It is favorable when the at least one actuating member is designed to be deformable at least in sections, preferably even elastically deformable. As a result of deformation of the at least one actuating member which can be based on a material elasticity thereof, it is possible, for example, for the at least one actuating member to be movable at least in sections. The at least one actuating member can thus be arranged on the carrier element with an elastically deformable section and have a connecting element at its end facing away from the carrier element. This can then be moved relative to the carrier element as a result of the deformability of the actuating member. The deformable section of the at least one actuating member can, in particular, form a film hinge, with which the at least one actuating member is arranged on the carrier element so as to be pivotable.

In a further embodiment of the data carrier according to the invention, it may be provided for the at least one actuating member to be arranged on the at least one connecting element. For example, the at least one actuating member can, in this embodiment, be rigidly secured to the at least one connecting element which is arranged on the carrier element or mounted thereon so as to be elastically deformable. In this way, the at least one connecting element can be moved as a result of actuation of the actuating member.

A constructionally simple embodiment of the data carrier may be achieved in that the carrier element is of a plate-like design.

In a preferred embodiment of the data carrier according to the invention, it has proven to be favorable when the carrier element is deformable at least in sections such that the connecting device can be transferred from the connecting position into the release position. This embodiment is of advantage, in particular, when the carrier element comprises at least one connecting element. This enables the data carrier to have a simple construction.

For the same reason, it is of advantage in a further, preferred embodiment of the data carrier according to the invention when the carrier element limits the receptacle at least in sections. In this case, the receptacle is limited, for example, by the carrier element and at least one connecting element and is, in particular, defined between them.

As already mentioned, it is favorable when the connecting device is designed in such a manner that it can be transferred from the connecting position into the release position without any forces acting on the implant. It is likewise of advantage when the connecting device is designed in such a manner that it can be transferred from the release position into the connecting position without any forces acting on the implant. Therefore, forces acting only on the data carrier can be sufficient for its connection to the implant. As a result, the data carrier has an even better handling capability.

The data carrier preferably comprises a restoring member which is designed in such a manner that the connecting device takes up the connecting position automatically proceeding from the release position. The connecting position can define, for example, a rest or basic position of the connecting device. As a result, it can be ensured that the data carrier can be detached from the implant only by a user deliberately initiating the release procedure and overcoming the action of the restoring member. In addition, the connection of the data carrier to the implant may be facilitated with an embodiment of this type. Following the at least partial insertion of the implant into the receptacle, the connecting device transfers automatically from the release position into the connecting position as a result of the restoring member, without any effort on the part of the user. This makes the handling of the data carrier particularly user-friendly.

The data carrier preferably comprises a pretensioning element, the implant being held in the receptacle in the connecting position contrary to its pretensioning. This makes it possible to hold the implant in the receptacle in a particularly reliable manner. The user must first overcome the action of the pretensioning element in order to transfer the connecting device from the connecting position into the release position. It is possible, in particular, for the pretensioning element to be identical to the restoring member described above. This enables the data carrier to have a particularly simple construction.

The data carrier advantageously comprises a data storage device for storing data which can identify the implant. The data suitable for identifying the implant can, for example, include the name of the producer, type and size of the implant, article number, serial number, batch number or the like. As a result, it can be determined, for example, what type of implant is involved. When a serial number is present, a precise identification of the implant can even be carried out. This of great importance for documentation purposes for medical procedures and can serve the purpose of tracing the implant after its insertion.

It may be provided for the data carrier to be designed such that the data are stored in a physical form. It is possible, for example, to apply the data by way of embossing on the data carrier or by spraying onto the data carrier. A utilization of the data storage device, with which the storing of data can be carried out by way of writing on the data carrier, application of labels, tags or the like, is also understood in the present case as a suitable way of storing data in a physical form.

Furthermore, the data storage device can be designed such that the data suitable for identifying the implant are stored in an electronic form. In this respect, it may be provided for the data stored in such a manner to be modifiable and, in particular, modifiable one time.

It is favorable when the data storage device can be read in a non-contact manner. For example, it may be read inductively with the use of a transponder, in particular, an RFID chip. This is of significance, especially in the case of data stored electronically.

Furthermore, the data carrier can be read in a non-contact manner as a result of the use of optical means. This is understood in the present case as the reading of the data stored by the data storage device with the aid of an optical imaging device, such as, in a particularly advantageous manner, the human eye. Optical scanners and/or bar code readers, by means of which the data stored in the data storage device can be read, are also conceivable.

It is favorable for a simplified and fast reading of the data when the data storage device can be read mechanically, i.e., with the aid of a technical device.

The data storage device is advantageously arranged, at least partially, on the carrier element since, as a result, it is possible to give the data carrier a compact constructional form.

It has proven to be favorable when the data carrier is produced at least partially from a plastic material. This allows a technically simple and inexpensive production of the data carrier. Polymer materials, such as polyether ether ketone (PEEK), polyether ketone ketone (PEKK), polyoxymethylene (POM), polytetrafluoroethylene (PTFE, Teflon) or the like, are, for example, conceivable.

It is of advantage when the data carrier is produced at least partially from a shape memory metal alloy. In this respect, the movable parts of the data carrier are preferably manufactured from a shape memory metal alloy. Such an embodiment is useful, in particular, when the data carrier is reused. It can be ensured that the data carrier can again take up a defined original shape even after several movement cycles.

It has proven to be favorable when the data carrier is produced from a sterilizable material since this allows it to be sterilized. The data carrier can advantageously be sterilized as often as required. The sterilization is preferably brought about in a single procedure with the implant held in the receptacle. As a result of the sterilization of the data carrier, a risk of infection for the patient, for whom the implant is intended, can be reduced even when the data carrier is intended to remain in the body of the patient.

It is of advantage when the data carrier is produced at least partially from a material which has a thermal coefficient of expansion which is greater than the thermal coefficient of expansion of titanium or that of an alloy, the main component of which is titanium. Medical implants for the fixing of bones or bone fragments are normally produced from titanium or a titanium alloy, the main component of which is titanium. It is possible by means of this embodiment for the sections of the data carrier limiting the receptacle to expand to a greater extent than the implant during common sterilization of the data carrier and an implant held in the receptacle. In the case where the implant is seated in the receptacle in a form-locking manner at a normal temperature, spaces may be formed between the implant and the specified sections due to heating up during the sterilization procedure. Hot steam can penetrate the spaces during the sterilization procedure, cover the areas of the implant arranged in the receptacle and likewise sterilize them.

The data carrier is preferably produced from a resorbable material. Normally, the data carrier is to be detached from the implant as determined after the insertion of the implant into the body of the patient. If this does not occur intentionally or also unintentionally, the risk of an infection for the patient as a result of the data carrier remaining in the body can be reduced in this way.

It is of advantage when the data carrier can be detached from the implant free from residue since, as a result, it can be ensured that no residues of the data carrier, which represent a potential risk of infection for the patient, remain during the insertion of the implant into the body of the patient and detachment of the data carrier from the implant.

It is favorable for the inexpensive production of the data carrier when the data carrier is designed in one piece. Preferably, it is produced, in particular, in one piece from a plastic material.

As already mentioned, the invention also relates to a surgical implantation system.

Moreover, in accordance with the invention, it is suggested in a generic surgical implantation system, that the data carrier has an actuating device which can be actuated by a user and with which the connecting device can be transferred from a connecting position, in which the implant is held in the receptacle, into a release position, in which the data carrier can be detached from the implant.

The implantation system according to the invention has the advantages already described in conjunction with the explanations concerning the data carrier according to the invention.

It is of advantage, in particular, when the data carrier is designed as one of the data carriers described above. The implantation system then has the additional advantages mentioned in the explanations concerning these data carriers.

The following description of preferred embodiments serves to explain the invention in greater detail in conjunction with the drawings.

### BRIEF DESCRIPTION OF THE DRAWINGS

FIG. 1: shows a perspective view of a preferred embodiment of a surgical implantation system according to the invention, comprising a preferred embodiment of a surgical data carrier according to the invention and an implant connected to it and

FIG. 2: shows a view similar to FIG. 1 during the connection of the implant to the surgical data carrier.

### DETAILED DESCRIPTION OF THE INVENTION

A preferred embodiment of an implantation system according to the invention is given the reference numeral 10 in the drawings. The implantation system 10 comprises a medical implant 12 as well as a preferred embodiment of a surgical data carrier 14 likewise according to the invention.

The implant 12 is designed as a surgical perforated plate 16 produced from titanium for the connection of bones and/or bone fragments. The perforated plate has the shape of a T with a longitudinal section 18 which corresponds to the long stroke of the T as well as with a transverse section 20 which is arranged at one end of the longitudinal section 18, is aligned at right angles to it and corresponds to the cross stroke of the T. A plurality of screw holes 22, 24, 26, 28 and 30 in the form of passages in the longitudinal section 18 and the transverse section 20 can have securing means which are not shown in the drawings, such as, for example, bone screws, passing through them in order to attach the implant 12 as predetermined to a bone and/or bone fragment. The screw hole 22 is located in the center of the transverse section 20, at the intersection thereof with an imaginary extension of the longitudinal section 18.

The perforated plate 16 is, altogether, of a flat design and approximately defines a perforated plate plane.

The data carrier 14, which forms the implantation system 10 together with the implant 12, is produced in one piece from a material which can be sterilized in saturated steam as often as required. This can, for example, be a polymer material, such as, for example, polyether ether ketone (PEEK), polyether ketone ketone (PEKK), polyoxymethylene (POM), polytetrafluoroethylene (PTFE, Teflon) or the like.

A carrier element 32 of the data carrier 14 is configured as a carrier plate 34 and has an approximately rectangular cross section in a plan view of the data carrier. Two elongated arms 36 and 38, which are spaced from one another and form a space 40 between them, project from the data carrier in a web-like manner.

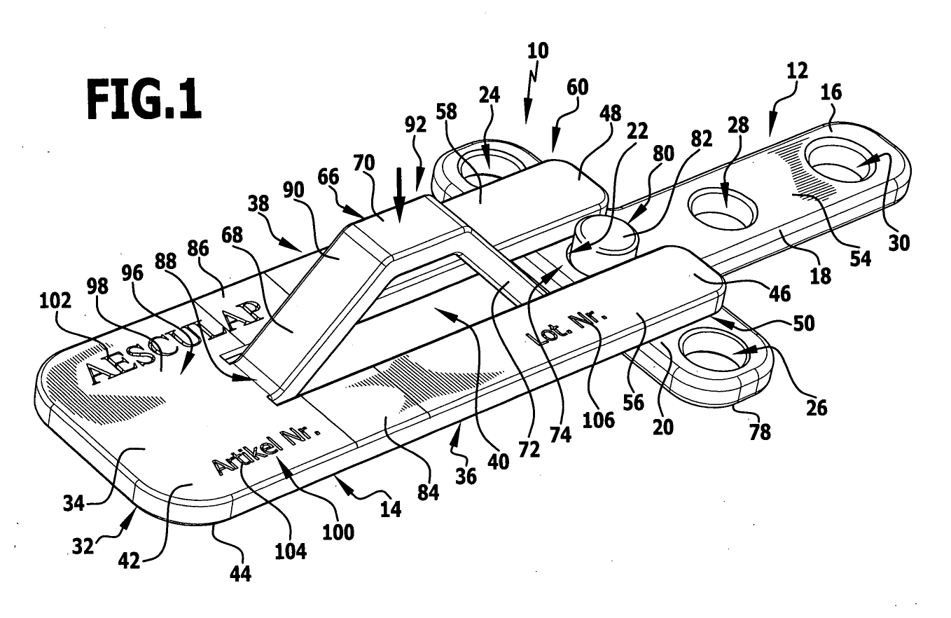

In the representation of the data carrier 14 shown in FIG. 1, the arms 36 and 38, which are of a flat design, extend in a carrier plate plane defined by the carrier plate 34. The carrier plate 34 and the arms 36 and 38 therefore form approximately the shape of a long U. With respect to a plane of symmetry which runs at right angles to the plane of the carrier plate and parallel to the direction of the arms 36 and 38 through the center of the space 40, the data carrier is, altogether, of a symmetrical design.

The carrier plate 34 as well as the arms 36 and 38 have a common upper side 42 and a common underside 44. On the underside 44, the arms 36 and 38 have contact surfaces 50 and 52, respectively, at their arm ends 46 and 48, respectively, facing away from the carrier plate 34.

The data carrier 14 can abut on the perforated plate 16 via the contact surfaces 50 and 52. In particular, the data carrier 14 can abut via them on the upper side 54 of the perforated plate 16 in the area of the transverse section 20. The arms 36 and 38 can, in this respect, be placed on either side of the screw hole 22 so that the screw hole 22 is arranged in the space 40 between the arms 36 and 38. Furthermore, the data carrier 14 can abut on the implant 12, in particular, such that the plane of the carrier plate is aligned parallel to the plane of the perforated plate (FIG. 1).

As a result of the possibility of the arms 36 and 38 abutting on the implant 12, they form a first connecting element 56 and a second connecting element 58 which are part of a connecting device 60 comprised by the data carrier 14 for the connection of the data carrier 14 to the implant 12.

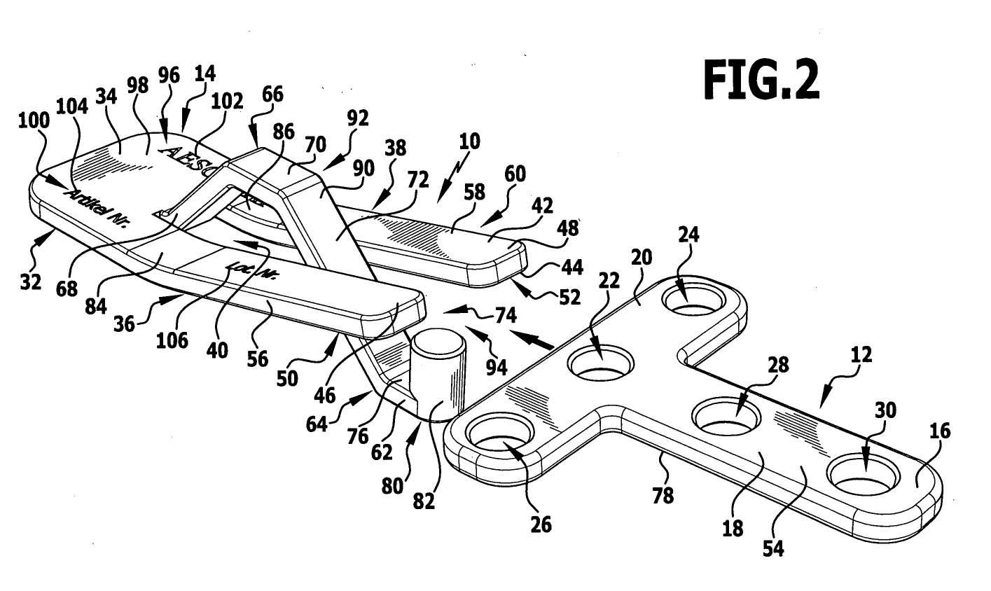

An additional, third connecting element 62 of the connecting device 60 (FIG. 2) is formed at a free end 64 of an arm 66 which is arranged on the carrier plate 34 with its end facing away from the third connecting element 62.

The arm 66 contacts the carrier plate 34 in the space 40 between the arms 36 and 38. It rises out of the plane of the carrier plate close to the carrier plate 34 with a first segment 68; a second segment 70 runs parallel to the plane of the carrier plate and a third segment 72, at the end of which the third connecting element 62 is formed, intersects the plane of the carrier plate. In this way, a receptacle 74 for the implant 12 is formed between the first connecting element 56 and the second connecting element 58, on the one hand, as well as the third connecting element 62, on the other hand, for connecting the implant to the data carrier 14.

Apart from the first connecting element 56 and second connecting element 58 already mentioned, the third connecting element 62 also abuts on the perforated plate 16 when a connection between the data carrier 14 and the perforated plate 16 is present. The third connecting element 62 has for this purpose a contact surface 76 which can abut on an underside 78 of the perforated plate at the transverse section 20 close to the screw hole 22. In this way, the implant 12 can be held in the receptacle 74 with its transverse section 20 between the first connecting element 56, the second connecting element 58 and the third connecting element 62.

In order to hold the implant 12 even more reliably in the receptacle 74, the connecting device 60 has a securing member 80 in the form of a bearing pin 82. This is designed to project from the third connecting element 62 in a direction at right angles to the plane of the carrier plate. In the connecting position of the connecting device 60, the bearing pin 82 can engage through the screw hole 22 so that the perforated plate 16 is held in the receptacle 74 in a particularly reliable manner.

In addition, the fact that the arm 66 is designed to be pretensioned in relation to the arm 36 and the arm 38 at least in the connecting position has a beneficial effect for the reliable holding of the perforated plate 16 in the receptacle 74. Consequently, the first connecting element 56, the second connecting element 58 and the third connecting element 62 act in the connecting position of the connecting device 60 as clamping elements which clamp the implant 12 between them in the connecting position so that this is held in a force-locking manner in the receptacle 74. The arm 66 therefore forms an elastic element, contrary to the pretensioning of which the implant 12 is held in the receptacle 74 in the connecting position and which can be overcome in the manner described below in order to detach the data carrier 14 from the implant 12.

In further, preferred embodiments of the data carrier according to the invention, the connecting elements used can be designed, for example, as snap-in elements.

An alternative securing member could be configured such that it forms a recess for an implant held in the receptacle and engages around or behind it. A design of a securing member as a snap-in nose which engages in a complementary snap-in receptacle of an implant is also possible.

The first connecting element 56, the second connecting element 58 and the third connecting element 62 are movable relative to one another. In order to make the relative movement possible, the arm 36 and the arm 38 each have an area of deformation 84 and 86, respectively, via which the arms 36 and 38, respectively, are arranged on the carrier plate 34. The areas of deformation 84 and 86 are elastically, i.e., reversibly deformable, wherein the arms 36 and 38 are arranged in the plane of the carrier plate in their basic position (FIG. 1).

The arm 66 likewise has an area of deformation 88, via which it is arranged on the carrier plate 34 with its first segment 68. The area of deformation 88 is likewise reversibly deformable and, as already mentioned, designed such that the arm 66 forms a pretensioning element in relation to the arms 36 and 38 in the connecting position of the connecting device 60.

The areas of deformation 84, 86 and 88 form in this way film hinges and so the arms 36, 38 and 66, respectively, are arranged on the carrier plate 34 so as to be pivotable.

The procedure for detaching the data carrier 14 from the implant 12 can be as follows:

The arm 66 can be acted upon, preferably at its second segment 70, with a releasing force directed onto the carrier plate 34 (symbolized in FIG. 1 by the arrow) and a contrary holding force can be exerted on the arms 36 and 38 from the underside 44 in a direction contrary to the releasing force. As a result, the arms 36, 38 and 66 are deformed at their areas of deformation 84, 86 and 88, respectively, which form the film hinges, wherein they are each pivoted parallel to or in the plane of symmetry in relation to the carrier plate 34. The first connecting element 56 and the second connecting element 58 can, therefore, be moved in relation to the third connecting element 62 and, in particular, be moved in a direction pointing away from it. Consequently, the receptacle 74 can be enlarged to such an extent that the bearing pin 82 can be removed from the screw hole 22. This defines a release position of the connecting device 60, in which the data carrier 14 can be detached from the implant 12. FIG. 2 shows the data carrier 14 with the connecting elements 56, 58 and 62 moved relative to one another.

The arm 66 therefore forms an actuating member 90 of an actuating device 92 which is comprised by the data carrier 14 and by means of which the connecting device 60 can be transferred from the connecting position into the release position. The data carrier 14 or rather the implantation system 10 is, therefore, user-friendly and can be handled in a simple manner.

In addition, the transfer of the connecting device 60 can be carried out by a user in a controllable manner. This reduces the risk of the data carrier 14 being detached from the implant 12 in an inappropriate manner which could lead to damage to the data carrier 14. As a result of such damage it would be possible for fragments of the data carrier 14 to remain in the body of the patient and represent, in this case, a potential risk of infection. This risk is reduced by the implantation system 10 according to the invention. The data carrier 14 can be detached from the implant 12, in particular, free from residue.

The connecting device 60 can be transferred from the connecting position into the release position without any forces acting on the implant 12. This gives the data carrier 14 and the implantation system 10 an improved handling capability because no contrary holding force acting on the implant 12 need be applied in order to detach the data carrier 14 from the implant 12.

The procedure for connecting the data carrier 14 to the implant 12 can be as follows:

The connecting device 60 can be transferred by means of the actuating member 90, as described above, into the release position, in which the receptacle 74 is enlarged to such an extent that the implant 12 can be inserted into the receptacle 74 via an insertion opening 94 (symbolized by the arrow in FIG. 2). Subsequently, the bearing pin 82 can be arranged in the screw hole 22. As a result of the elastic deformability of the areas of deformation 84, 86 and 88, the connecting device 60 takes up the connecting position automatically proceeding from the release position. In this way, the areas of deformation 84, 86 and 88 together form a restoring member of the data carrier 14.

Alternatively, a restoring member could be designed, for example, as a tension spring tensioned between the arm 66 and the arms 36 or 38 or between the arms 36 and 38.

The connecting device 60 is, therefore, of such a design that it can be transferred from the release position into the connecting position without any forces acting on the implant 12. The data carrier 14 and, with it, the implantation system 10 therefore have a particularly simple handling capability.

Variations of the data carrier 14 described above have arms 36 and 38 which are rigidly secured to the carrier plate 34 and comprise no areas of deformation 84 and 86. Because the arm 66 does, however, have an area of deformation 88, the mode of operation of such a data carrier does not differ substantially from that of the data carrier 14 and so the same advantages can be achieved with it.

The implantation system 10 can be grasped by a user, for example, a surgeon at the data carrier 14 and, for example, moved, transported or the like to its predetermined location. In this way, the data carrier 14 forms a handling device for the implant 12. The predetermined location is, for example, a bone or bone fragment which is to be screwed to the implant 12.

On its upper side 42, the data carrier 14 has a data storage device 96 with a storage area 98 which partially comprises the upper side 42 in the area of the carrier plate 34 but also the upper side 42 in the area of the arms 36 and 38. Data 100, schematically represented in the drawings in part by place holders, are deposited at the storage area 98. These data 100 identify and characterize the implant 12 and include, for example, the name of the producer, the batch number, the article number, the serial number, the type and size of the implant and the like.

The data 100 are cast directly onto the data carrier 14 during its production and protrude from the carrier plate 34 as well as the arms 36 and 38 as relief-like writings 102, 104 and 106. The data 100 can be read optically, i.e., with the aid of an optical imaging device, in particular, the human eye. They can, therefore, be recognized by a user, such as, for example, a surgeon.

The data carrier 14 serves the purpose of describing, marking and identifying the implant 12 by means of the data 100. After the implant 12 has been inserted into the body of a patient, the data carrier 14 can be detached from the implant 12 as explained above and remain in the patient's file, for example, for documentation purposes so that it is possible to trace the implant 12 back.

Different types of embodiments of a data carrier according to the invention comprise data storage devices, with which the data are deposited in a different physical form to that of reliefs, for example, by way of embossing, attachment of an adhesive label or tag, lettering or printing on the surface or the like. Optical readability of the data can also be achieved by means of a mechanically operated, optical imaging device. The use of a bar code reader, with which bar codes can be read which are advantageously arranged on an adhesive label secured to the carrier plate 34 and/or the arms 36 and 38, is mentioned as an example.

In the case of the data carrier 14, the data storage device 96 can also be of an electronic nature so that the data can be stored in an electronic manner and/or also computed in the storage device. In particular, it may be provided for the electronic storage device to be read inductively and, therefore, without contact, for example, with the use of a transponder and particularly an RFID chip. An electronic RFID chip can preferably be arranged, for example, in the carrier plate 34.

As likewise mentioned at the outset, the data carrier 14 is produced from a sterilizable plastic material. This has a thermal coefficient of expansion which is greater than the thermal coefficient of expansion of titanium, from which the implant 12 is produced. During the sterilization of the implantation system 10, gaps can be formed during heating between the first connecting element 56, the second connecting element 58, the third connecting element 62 and the implant 12, in particular, its transverse section 20 on account of the increased thermal coefficient of expansion. This allows the implant 12 to be sterilized at the transverse section 20, as well, without it being necessary to separate the data carrier 14 from the implant 12 beforehand. This gives the implantation system 10 a great user-friendly capability.

1. Surgical data carrier for the identification of a medical implant, in particular, a surgical plate for the fixing of bones or bone fragments, wherein the data carrier has a carrier element and a connecting device comprising a receptacle for the insertion of at least part of the implant, wherein the data carrier has an actuating device actuatable by a user, the connecting device being transferable with said actuating device from a connecting position, the implant being held in the receptacle in said connecting position, into a release position, the data carrier being detachable from the implant in said release position.

2. Surgical data carrier as defined in claim 1, wherein the connecting device is designed in such a manner that it is transferable from the connecting position into the release position without any forces acting on the implant.

3. Surgical data carrier as defined in claim 1, wherein the receptacle is enlarged during the transfer of the connecting device from the connecting position into the release position.

4. Surgical data carrier as defined in claim 1, wherein the connecting device comprises at least one connecting element limiting the receptacle at least in sections and interacting with the implant for the connection of the data carrier.

5. Surgical data carrier as defined in claim 4, wherein the carrier element comprises the at least one connecting element.

6. Surgical data carrier as defined in claim 4, wherein the at least one connecting element) is arranged on the carrier element.

7. Surgical data carrier as defined in claim 4, wherein the at least one connecting element has a contact surface abutting on the implant in the connecting position.

8. Surgical data carrier as defined in claim 4, wherein the connecting device comprises two or more connecting elements limiting the receptacle in sections.

9. Surgical data carrier as defined in claim 8, wherein the two or more connecting elements are movable relative to one another.

10. Surgical data carrier as defined in claim 6, wherein the at least one connecting element is arranged or mounted on the carrier element so as to be movable.

11. Surgical data carrier as defined in claim 5, wherein the at least one connecting element is designed to be deformable at least in sections.

12. Surgical data carrier as defined in claim 5, wherein the at least one connecting element is designed as a snap-in element.

13. Surgical data carrier as defined in claim 5, wherein the at least one connecting element is designed as a clamping element.

14. Surgical data carrier as defined in claim 1, wherein the connecting device has a securing member for securing the implant in the receptacle in the connecting position.

15. Surgical data carrier as defined in claim 14, wherein the securing member is adapted to be brought into engagement with a receptacle comprised by the implant.

16. Surgical data carrier as defined in claim 1, wherein the actuating device has at least one actuating member for moving at least one connecting element comprised by the connecting device.

17. Surgical data carrier as defined in claim 16, wherein the at least one actuating member comprises the at least one connecting element.

18. Surgical data carrier as defined in claim 16, wherein the at least one actuating member is arranged on the carrier element.

19. Surgical data carrier as defined in claim 16, wherein the at least one actuating member is arranged or mounted on the carrier element so as to be movable.

20. Surgical data carrier as defined in claim 16, wherein the at least one actuating member is deformable at least in sections.

21. Surgical data carrier as defined in claim 16, wherein the at least one actuating member is arranged on the at least one connecting element.

22. Surgical data carrier as defined in claim 1, wherein the carrier element is of a plate-like design.

23. Surgical data carrier as defined in claim 1, wherein the carrier element is deformable at least in sections such that the connecting device is transferable from the connecting position into the release position.

24. Surgical data carrier as defined in claim 1, wherein the carrier element limits the receptacle at least in sections.

25. Surgical data carrier as defined in claim 1, wherein the connecting device is designed in such a manner that it is transferable from the release position into the connecting position without any forces acting on the implant.

26. Surgical data carrier as defined in claim 1, wherein the data carrier comprises a restoring member designed in such a manner that the connecting device takes up the connecting position automatically proceeding from the release position.

27. Surgical data carrier as defined in claim 1, wherein the data carrier comprises a pretensioning element, the implant being held in the receptacle in the connecting position contrary to the pretensioning of said element.

28. Surgical data carrier as defined in claim 1, wherein the data carrier comprises a data storage device for storing data able to identify the implant.

29. Surgical data carrier as defined in claim 28, wherein the data storage device is adapted to be read in a non-contact manner.

30. Surgical data carrier as defined in claim 28, wherein the data storage device is arranged at least partially on the carrier element.

31. Surgical data carrier as defined in claim 1, wherein the data carrier is produced at least partially from a plastic material.

32. Surgical data carrier as defined in claim 1, wherein the data carrier is produced at least partially from a shape memory metal alloy.

33. Surgical data carrier as defined in claim 1, wherein the data carrier is produced from a sterilizable material.

34. Surgical data carrier as defined in claim 1, wherein the data carrier is produced at least partially from a material having a thermal coefficient of expansion greater than the thermal coefficient of expansion of titanium or that of an alloy having titanium as its main component.

35. Surgical data carrier as defined in claim 1, wherein the data carrier is produced from a resorbable material.

36. Surgical data carrier as defined in claim 1, wherein the data carrier is detachable from the implant free from residue.

37. Surgical data carrier as defined in claim 1, wherein the data carrier is designed in one piece.

38. Surgical implantation system, comprising at least one medical implant, in particular, a surgical plate for the fixing of bones or bone fragments and a surgical data carrier detachably connectable to the implant for the identification of the implant, wherein the data carrier has a carrier element and a connecting device comprising a receptacle for the insertion of at least part of the implant, wherein the data carrier has an actuating device actuatable by a user, the connecting device being transferable with said actuating device from a connecting position, the implant being held in the receptacle in said connecting position, into a release position, the data carrier being detachable from the implant in said release position.

39. Surgical implantation system as defined in claim 38, wherein the connecting device is designed in such a manner that it is transferable from the connecting position into the release position without any forces acting on the implant.
